(19) United States
(12) Patent Application Publication
(10) Pub. No.: US 2007/0135768 A1
(43) Pub. Date: Jun. 14, 2007

(54) Spinal and epidural regional anesthesia catheter

(76) Inventor: Wayne D. Carlsen, Riverton, UT (US)

Correspondence Address: KIMBERLY-CLARK WORLDWIDE, INC., 401 NORTH LAKE STREET, NEENAH, WI, 54956, US

(21) Appl. No.: 11/302,643

(22) Filed: Dec. 14, 2005

(51) Int. Cl. A61M 5/178

(52) U.S. Cl. 604/158

(57) ABSTRACT

A system for administering regional anesthesia to a patient contains a regional anesthesia catheter and a partially splittable needle. The catheter has a first length and a second length, the first length having a first inner diameter, a first outer diameter, and a wall thickness, the second length having a first inner diameter, a first outer diameter, and a wall thickness, the diameters and the wall thickness of the second length being less than those of the first length. The needle is an atraumatic needle splittable along its length but remains hinged together by the needle tip itself. The system is particularly adapted for epidural and spinal catheterization with a medicament.

### BACKGROUND

The present invention relates generally to the field of regional anesthesia catheters such as epidural and spinal catheters as well as to methods of using the same. More specifically, the invention provides a system for continuously administering an epidural or spinal anesthetic or analgesic agent to a patient that overcomes many of the difficulties associated with present day catheters and procedures for their placement.

Spinal catheters are designed to be placed in the intrathecal space, or that space within the spinal column under the arachnoid membrane of the brain or spinal cord also known as the subarachnoid space. This space is encased in a sheath known as the dura mater or dura which is the outermost and toughest membrane encasing the spinal cord. The intrathecal space is bathed in cerebrospinal fluid.

To introduce such a catheter into the intrathecal space, a spinal needle is utilized. Spinal needles generally are desired to have atraumatic tips that are intended to part or separate tissue as the needle passes through in lieu of being sharp and cutting through tissue planes. Often such needles are configured as a pencil point design with side ports. The side ports are typically ramped for ease in catheter placement as the catheter exits the port. As such, the ramp and port are designed to guide the catheter through the side of the needle when the catheter is threaded through the needle and ultimately direct the catheter to the desired location.

More specifically, the needle is used to guide the catheter into the intrathecal or subarachnoid space by introducing the catheter through a central bore of the needle located at the needle's central longitudinal axis. Following the introduction of the catheter into the needle, the needle is inserted into the patient's back and situated so that the distal end of the needle punctures the dura and enters the intrathecal space. In many procedures, the needle is introduced into the patient's back in a similar manner as described above but it is done prior to threading the catheter though the bore of the needle. In any event, after placement, the needle is withdrawn from the patient's back by sliding it over the catheter and removing it from the proximal end of the catheter while the distal end of the catheter remains in place in the intrathecal or subarachnoid space.

For example in a typical procedure, an introducer needle if used by the physician is placed through the skin into the patient's back. Next, the spinal needle with a stylet situated in its central bore is passed through the introducer needle or directly introduced into the patient's back through the remaining tissue and tendon planes, through the dura mater and ultimately into the subarachnoid space. The stylet is removed and cerebral spinal fluid is either observed leaking out of the needle or drawn through the needle with a syringe to ensure that the proper anatomical space is located. The catheter is next passed through the needle into the subarachnoid space as stated above.

While preventing the catheter from being drawn out of the subarachnoid or intrathecal space, the spinal needle and introducer needle, if present, are removed from the patient's back and passed over the catheter until removed from the catheter at the proximal end. Once the needle is removed, an adapter is attached to the catheter tubing by threading the proximal end of the catheter into or through the adapter. In many embodiments the adapter is axially rotated such that a grommet is tightened against the outside of the catheter until a secure seal is formed. The adapter is subsequently used to attach the catheter to an infusion pump or syringe which is used to introduce a medicament into the intrathecal space. The adapter itself is normally secured to the patient's body, typically by tape, so that it does not interfere with patient comfort, patient movement, or is inadvertently dislodged. The catheter is secured and kept clean with tape at the insertion site to prevent it from being dislodged from the intrathecal space.

The problems faced by the practitioner in this procedure are that the pressure of the cerebrospinal fluid is greater than atmospheric pressure and thus the fluid will flow out of the intrathecal space when the dura is breached or punctured. Loss of cerebrospinal fluid may cause pressure changes in the intrathecal cavity and such pressure changes may induce severe post dural puncture headaches. As a consequence any needle that is introduced into the intrathecal space should be small so as to minimize cerebrospinal fluid loss and the resulting pressure changes.

To accommodate small needles, it is necessary for the catheter to have a small diameter as well, for the same reasons. Moreover, the tip of the needle used to introduce the catheter into the intrathecal space should be designed to minimize any cutting of the dura which could lead to the same patient discomfort and consequences. Due to these requirements, in practice, the physician faces a number of difficulties. First, it is difficult to infuse medication through a long narrow catheter. High infusion pressures are required. Second, it is difficult to handle or manipulate a small slender catheter as it is more prone to damage, kinking or breaking. Moreover, it is challenging to secure such a small catheter in a manner that provides strain relief, prevents inadvertent withdrawal, and prevents kinking. Due to the infusion pressures required with a small catheter, the catheter's length is limited which makes it more difficult to place the adapter in a location where patient comfort can be achieved.

Although cerebral spinal fluid can be drawn from the needle to ensure proper placement, cerebral spinal fluid cannot be drawn adequately through a long catheter to ensure that proper placement is maintained because the pressure drop required to pull the cerebral spinal fluid through the catheter cannot be achieved. It is understood by those skilled in the art that the maximum achievable pressure is the difference between the pressure in the intrathecal space and a complete vacuum and this is not enough pressure differential to draw the cerebral spinal fluid through the catheter in a reasonable amount of time for placement evaluation. In addition, with a small diameter catheter, it is difficult to attach the adapter to the catheter in a manner that maintains a seal between the adapter and the catheter and secures the catheter without crushing or collapsing the catheter.

### SUMMARY

Various objects and advantages of the invention will be set forth in part in the following description, or may be apparent from the description, or may be learned from practice of the invention.

A system for administering regional anesthesia to a patient would contain a regional anesthesia catheter, a partially splittable needle, and an adapter for attaching the catheter to a source of medication. The catheter would have a first length and a second length. The first length has a first inner diameter, a first outer diameter, and a wall thickness. The second length has a first inner diameter, a first outer diameter, and a wall thickness. The diameters and the wall thickness of the second length are characterized in that they are less than those of the first length so as to form “bump” tubing.

In some embodiments, the second length of the catheter is as short as possible so as to minimize resistance to the flow of liquid therethrough. For example, the first length may be about two to about five times the length of the second length. In some embodiments it may be about three and one-half times the length of the second length.

The partially splittable needle has a hollow shaft having opposed distal ends and proximal ends. The distal end has a tip for insertion into a patient's spinal area. The needle contains a pair of opposed wings at the proximal end adapted to be grasped by a user for splitting the needle longitudinally. The needle is etched or perforated longitudinally along the needle's axis and the perforation terminates at a side port which forms a distal terminus of a cannula through the central bore of the needle.

The needle may also have a split guide hole through the needle proximate to the distal end of the needle where the perforation terminates so that the needle after being split remains hingedly connected by the tip. The needle may also have a ramped side port proximal to the distal end leading from a central bore in the hollow shaft to an exterior surface on the needle. Such a ramped side port is to direct the catheter from the needle to the desired location within the patient. The needle may also have an atraumatic tip such as a pencil point type tip, two such examples being but not limited to a Sprotte type tip and/or a Whitacre type tip.

The system is particularly useful for the use of administering a spinal or an epidural anesthetic or analgesic agent.

Aspects of the invention will be described in greater detail below by reference to particular embodiments illustrated in the figures.

### BRIEF DESCRIPTION OF THE DRAWINGS

FIG. 1 is a perspective view of a regional anesthesia catheter in accordance with the invention.

FIG. 1a is a cross-sectional view of the transition section of the regional anesthesia catheter of FIG. 1.

FIG. 2 is a diagrammatical view of a needle and stylet for use in the FIG. 1 apparatus.

FIGS. 3a through 3e are close up views of various needle tips for use on the FIG. 2 needle.

FIGS. 4a and 4b are a diagrammatical view of the needle tip and the needle tip depicting the FIG. 1 catheter exiting therefrom.

### DETAILED DESCRIPTION

Reference will now be made in detail to embodiments of the invention, one or more examples of which are illustrated in the drawings. Each example is provided by way of explanation of the invention, and is not meant as a limitation of the invention. For example, features illustrated or described as part of one embodiment may be used with another embodiment to yield still a third embodiment. It is intended that the present invention include these and other modifications and variations.

As used herein, “proximal” refers generally to the direction towards a medical caregiver. “Distal” refers generally to the direction towards a patient.

As heretofore mentioned, the present invention is directed to a regional anesthesia catheter such as an epidural or spinal catheter which may be introduced via a removable needle into the epidural or intrathecal space for administering anesthetic or an analgesic agent. The catheter portion of the invention is characterized, inter alia, in that it contains distinctly different size diameters, to accommodate this, the needle is made to be partially splittable to enable its removal from the catheter once the catheter is properly placed. The partially splittable needle may have a hub assembly characterized by having a component that facilitates insertion of the needle into the subarachnoid space without increasing the risk of premature unwanted splitting of the needle during its insertion.

Splittable needles are per se old in the art for other medical procedures, but to the Applicant's knowledge a partially splittable needle used in conjunction with a variable diameter catheter is not.

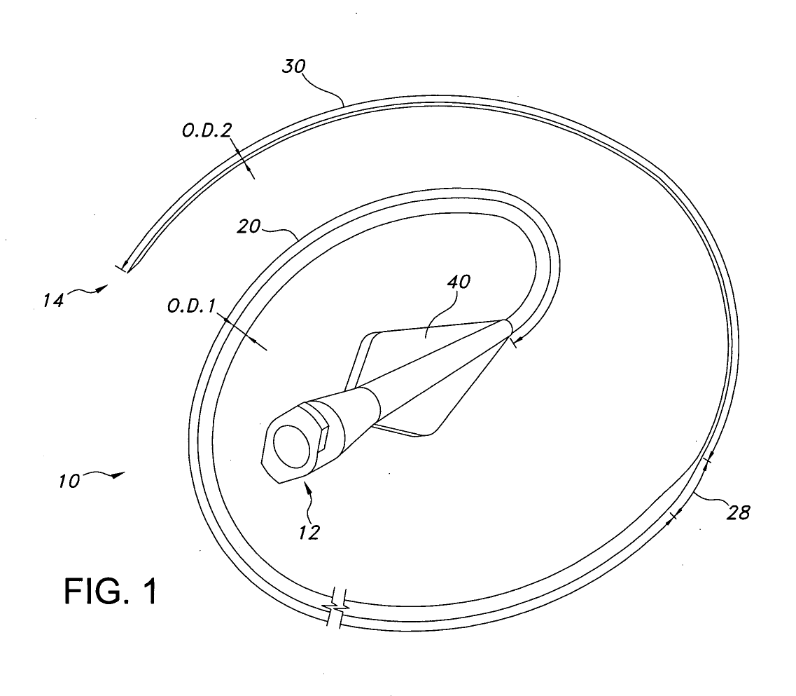

From this point, the invention will be described as a spinal catheter for use in administering a liquid, such as an anesthetic, an analgesic, or a diagnostic liquid into the intrathecal or subarachnoid space. However, using the device in the epidural or peridural space is considered to form a part of the invention as well. As such, as shown in FIG. 1, the spinal catheter 10 has a first or proximal end 12, a second or distal end 14, a first length 20, a second length 30, and a transition 28 between the first length 20 and the second length 30. Looking to FIG. 1a, it may be seen that the first length 20 has a first inner diameter 22, a first outer diameter 24, and a first wall thickness 26 whereas the second length 30 has a second inner diameter 32, a second outer diameter 34, and a second wall thickness 36. The second length 30 is characterized in that the inner diameter 32, the outer diameter 34, and the wall thickness 36 are each smaller in size than the corresponding component of the first length 20.

As may be seen in each figure, the transition 28 forms the region between the first and second lengths 20 and 30 respectively over which the change in size occurs. To create such a catheter 10, the tubing used to form the spinal catheter 10 is extruded or otherwise drawn through a die or other means for controllably stretching a portion of the tubing. The extrusion or drawing process enables one to create tubing or in this case a catheter having at least two differing regions characterized in that at least one region contains a larger original sized portion, i.e., the first length 20 which is transitioned into a second region which is a thinner, smaller diameter extruded portion, i.e., the second length 30. Of course this process would be known and understood by those of skill in the art and as such no need for additional explanation is necessary. Moreover, it would also be understood by those of skill in the art as to adaptation of the process for creating additional diameters should more than two diameters be desired. As such, such an embodiment is considered encompassed by the present invention as well.

This configuration overcomes many of the deficiencies discussed above which occur with the use of a smaller diameter catheter. For example, the resistance to flow of the liquid through the catheter is minimized, and the attachment and securement of the adapter to the catheter is improved and in some instances eliminated since an adapter combined at manufacture with the catheter may be used. The problems associated with the use of a pump or the supply of anesthetic or analgesic agent is minimized. Specifically, it is well known that smaller diameter tubing used for such things as spinal catheters have a high resistance to flow and as such require higher pressures to achieve an acceptable infusion rate of anesthetic or analgesic agent to the patient. The need for use of such higher pressures which challenge the pump performance as well as the security of the connection between the adapter and the tubing are minimized. Since the smaller diameters and thinner wall thicknesses associated with a smaller catheter make it difficult to attach an adapter during a procedure, this deficiency too is minimized as set forth below.

As stated, an adapter is typically attached to the catheter by threading the catheter through the adapter and then twisting or rotating a portion of the adapter until it clamps down and seals to the outside wall of the catheter. As also stated, it is difficult to accomplish this in a way that: (1) maintains the seal between the catheter wall and the adapter, (2) properly secures the adapter to the catheter, and (3) does not crush or otherwise collapse the catheter. Physicians and other medical personnel are familiar with such adapters and as such no further explanation of the procedures used to attach such adapters to the catheter 10 is considered necessary. It should be understood nonetheless that attaching such an adapter to the first length 20, i.e., that length of catheter 10 having the greater diameter would be desirable and would be far easier for the physician to accomplish.

Looking back to FIG. 1 however, another embodiment is depicted. This embodiment contains an adapter 40 permanently affixed to the catheter 10 thus freeing the physician from affixing the adapter to the catheter in its entirety. In this embodiment the adapter 40 would be formed on the catheter 10 during the manufacturing process itself. The adapter 40 is secured to the first length 20, i.e., the length having the greater diameter, at the proximal end 12. Proper selection of the catheter 10 would enable the second length 30 to be sized only as long as needed to ensure that a majority of the smaller diameter portion is inserted into the patient while the larger diameter remains outside the patient.

It should be apparent from the description of the needle below, that at least about a needle's length of the smaller diameter portion, i.e., the second length 30 would protrude from the patient's back so as to remove the needle from the catheter. By ensuring that the second length 30 is as short as possible, medication may be infused at lower pressures also ensuring that the pump performance is not compromised. Moreover, the catheter may be manipulated easier by the physician due to its larger size. Furthermore, this configuration also ensures that the smallest practical puncture is made into the patient thus minimizing trauma to the patient. For example, in some embodiments, the length 20 is about 2 to about 5 times that of the length 30, in other embodiments it may be about 3 times that of the length 30. In some embodiments, the ratios of L1 (referring to the length 20 and the transition region 28) to L2 (referring to the length 30) may be characterized by L2/L1≈0.3.

The procedure for administering a spinal anesthetic or analgesic agent in accordance with this invention will now be described in more detail.

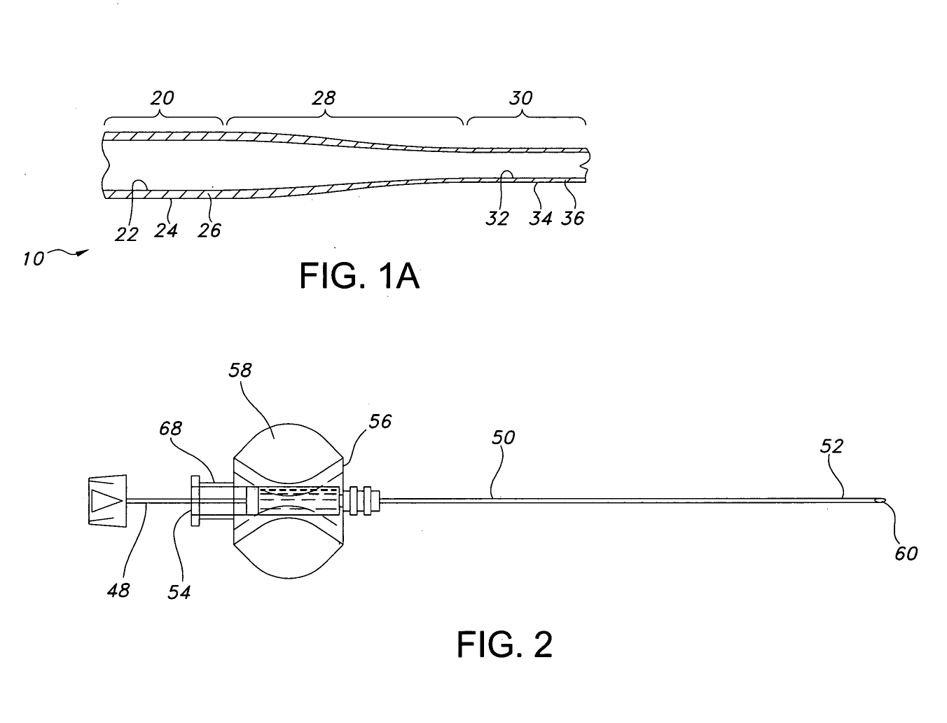

To administer the anesthetic or analgesic agent, great care must be taken in inserting a needle 50 such as that depicted in FIG. 2 into the patient's back to be sure it is properly positioned in the subarachnoid space. For this reason, the needle 50 may be calibrated, as shown, to assist the physician in determining the degree of insertion into the body. In a typical procedure, the patient, having been prepped for surgery, is brought into an induction room adjacent the operating room where the physician inserts the catheter 10 in preparation for the surgery. In an alternate procedure, the patient is in the process of child birth and the physician inserts the catheter 10 in preparation for the birthing process.

A local injection is first given to minimize pain and discomfort from introducing the needle 50. An introducer needle (not shown) may first be penetrated into the patient's back prior to introduction of the spinal needle 50. In the case of FIG. 2, however, a stylet 48, also optional, is shown as being partially inserted into the needle 50. The needle 50, and stylet 48 in the present case, is slowly and carefully inserted into the patient's back through the necessary tissue, tendon planes, and dura until it enters the intrathecal or subarachnoid space. Next, the stylet 48 is removed and the physician observes whether cerebral spinal fluid leaks out of the needle 50 or alternatively a syringe is affixed to the needle by virtue of the luer fitting 68. The syringe is used to draw fluid through the needle 50 for inspection and verification that it is cerebral spinal fluid, thus ensuring that the needle is indeed in the subarachnoid space. Next, the distal end 14 of the catheter 10 is inserted and fed into the needle 50 until the end 14 enters the intrathecal space.

While preventing the catheter 10 from being inadvertently withdrawn from the intrathecal space, the needle 50 is removed from the patient's back. Since the needle cannot be withdrawn from the patient and backed off the catheter due to the larger diameter length 20 in addition to the integral adapter 40, the needle must be at least partially splittable along its longitudinal axis. As such, once the needle 50 is removed from the patient's back, a portion of the needle adapted to initiate a split in the needle is grasped by the physician and manipulated until the needle is split and removed from the catheter 10.

If the catheter 10 is of the type that does not have an integral adapter 40 like the FIG. 1 depiction, a suitable adapter is attached to the proximal end 12 of the catheter 10. Finally, a syringe or infusion pump (not shown) is attached to the catheter 10 via the adapter 40 and medication is administered as appropriate. To ensure that the catheter 10 is secured in place to the patient's body, adhesive tape (not shown) is used to secure the catheter to the patient's skin in a location where it will not be in the way of the patient, unduly interfere with his or her movements, or be inadvertently removed from the patient.

Still looking to FIG. 2, it may be seen that the needle 50 has a distal or leading end 52 terminating in a tip 60 and a proximal or trailing end 54 at which a unitary hub assembly 56 is provided. The hub assembly 56 is preferably sufficiently transparent to permit visualization of any cerebral spinal fluid during the inspection and verification process described above thus confirming that the needle has entered the subarachnoid area. A pair of opposed wings 58 is located at the hub assembly 56. The wings 58 are provided for grasping and initiating a split along the length of the needle 50 so that it may be removed from the catheter 10, as will be described hereinafter.

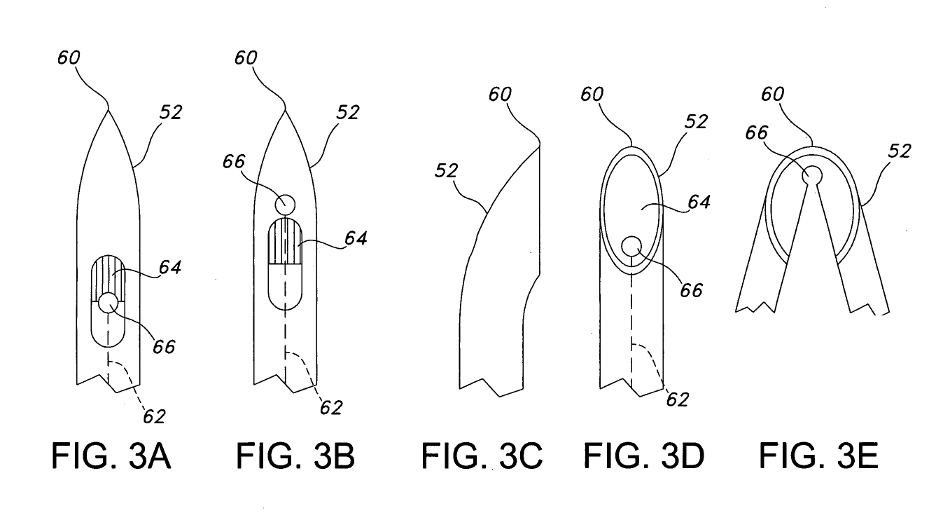

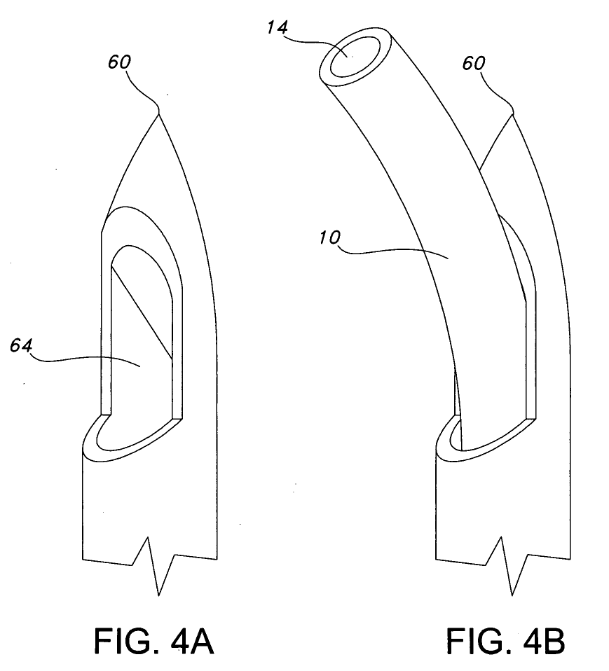

Looking now to various close-ups of the leading end 52 of the needle 50 as best seen in FIGS. 3a through 3e, it may be seen that the leading end 52 of the needle 50 terminates in a tip 60. The tip 60 may be configured into any number of possible atraumatic tip designs, for example Sprotte or Whitacre tip designs as shown in FIGS. 3a and 3b may be particularly suitable since they are atraumatic. Alternatively, Touhy type needles may also be used as depicted in FIGS. 3c through 3e and are particularly useful in epidural procedures. In any event, in looking to FIGS. 3a through 3e in combination with FIG. 2, it may be seen that a longitudinal slit 62 extends from the proximal end 54, through the hub assembly 56 and terminates at or proximate to a side port 64. The side port 64 forms the distal terminus of a cannula through the central bore of the needle 50 through which the catheter 10 exits the needle. The side port 64 may be ramped to direct the catheter 10 in its placement as shown in FIGS. 4a and 4b and enable it to enter the patient's body in the subarachnoid space.

Looking back to FIGS. 3a through 3e, a split guide hole 66 may be provided at the side port 64 as shown. As would be understood by those of skill in the art, the needle 50 would be gripped gently by the wings 58, as shown on FIG. 2 which may be repeatedly flexed, causing the needle 50 to open up along the longitudinal slit 62, thereby causing splitting or cracking along the longitudinal slit 62 which in turn causes the needle 50 to separate along the longitudinal slit 62 yet remain hingedly connected at the distal end 52. In function, the split guide hole 66 serves to stop the uncontrolled splitting of the needle 50 at the side port 64 while allowing removal of the needle 50 from the catheter 10. Additionally the side port 64 may be ramped as depicted in FIGS. 4a and 4b to direct the catheter in a desired direction upon the catheter's exit from the needle.

An advantage of having the needle 50 partially splittable is that a partially splittable needle allows the needle tip 60 to be designed in such a way that it has maximum or ideal atraumatic attributes. Specifically, the needle tip 60 does not contain any perforations or etching and as such does not form a jagged or ragged puncture hole in the dura. This leads to the ability of forming a needle having the ideal atraumatic point. It also prevents the needle tip from inadvertently splitting during the needle insertion process.

The needle may be perforated or etched by a laser or other means capable of perforating or etching the needle. An alternative could be that the needle blank itself may be continuously formed from flat sheet metal such as sections or from a roll. A groove of controlled depth is formed along the blank stock which is then rolled to a hollow configuration and then cut into individual needle barrels 50 with a longitudinal slit 62 along each needle barrel. The groove is oriented parallel to the slit 62 and along the opposite side of the barrel. Finally, the wings 58 are attached to the barrel on each side of the slit. The groove depth is sufficiently shallow to ensure adequate stiffness during use while being deep enough so that the needle 50 will split easily when the wings 58 are flexed, thereby separating the needle 50 from the catheter 10.

As is known by those of skill in the art, excellent stiffness, splitting and rolling properties are obtained with a sheet thickness of about 2-4 mils, a needle length of about 3.5″, about a 22 gauge barrel diameter, and a controlled groove depth about 50%.+−0.10% of the sheet thickness using a 304-316 stainless steel or equivalent.

The sheet stock may be in flat form such as in sections, say, ½-10 feet long, but is usually loaded on a roll. The open needle portion constitutes only about 7%-15% of the total barrel length, and this considerably improves the working strength of the needle. Thus, the needle of this invention can employ a relatively deep groove without losing structural integrity during use.

From the foregoing description it will thus be seen that the present invention solves the stated task of the invention in an elegant manner.

It will be appreciated that various changes may be made without departing from the scope of the invention herein contemplated.

For example, other splittable needle constructions may be readily suggested to those skilled in the art in the light of the foregoing description as will other designs for the guide means and/or the wing configuration for causing splitting of the needle.

In like manner, various changes in the adapter to which the spinal catheter is connected may be readily suggested to those skilled in the art.

Accordingly, all matter contained in the foregoing descriptions and shown in the accompanying drawings shall be interpreted as being illustrative and not in a limiting sense.

It should be understood that the present invention includes various modifications that may be made to the embodiments of the apparatus described herein as come within the scope of the appended claims and their equivalents.

What is claimed is:

1. A system for administering regional anesthesia to a patient comprising:
a regional anesthesia catheter having a first length and a second length, the first length having a first inner diameter, a first outer diameter, and a wall thickness, the second length having a first inner diameter, a first outer diameter, and a wall thickness, the diameters and the wall thickness of the second length being less than those of the first length;
an adapter for connecting to the first length, the adapter for introducing a liquid into the catheter from a liquid source;
a partially splittable needle comprising a hollow shaft having opposed distal ends and proximal ends, the distal end having a tip for insertion into a patient's spinal area.

2. The system of claim 1 comprising a spinal needle.

3. The system of claim 1 comprising an epidural needle.

4. The system of claim 1 wherein the needle comprises a ramped side port proximal to the distal end leading from a central bore in the hollow shaft to an exterior surface on the needle adapted to direct the catheter from the needle to the desired location within the patient.

5. The system of claim 1 wherein the needle comprises an atraumatic tip.

6. The system of claim 5 wherein the tip is a Sprotte type tip.

7. The system of claim 5 wherein the tip is a Whitacre type tip.

8. The system of claim 1 wherein the second length is as short as possible so as to minimize resistance to the flow of liquid therethrough.

9. The system of claim 1 wherein the adapter is integrated into the catheter at manufacture.

10. The system of claim 1 wherein the first length is about two to about five times the length of the second length.

11. The system of claim 1 wherein the first length is about three times the length of the second length.

12. The system of claim 1 wherein the needle comprises a pair of opposed wings at the proximal end adapted to be grasped by a user for splitting the needle longitudinally.

13. The system of claim 1 comprising a perforation or etching situated longitudinally along the needle's axis terminating at a side port which forms a distal terminus of a cannula through the central bore of the needle.

14. The system of claim 13 comprising a split guide hole forming a diametrical through hole proximate to the distal end of the needle where the perforation terminates.

15. The system of claim 14 wherein the needle after being split is hingedly connected by the tip.
